(19) United States
(12) Patent Application Publication
(10) Pub. No.: US 2007/0196550 A1
(43) Pub. Date: Aug. 23, 2007

(54) Edible product

(75) Inventor: Aharon Weissman, Misgav (IL)

Correspondence Address: FAY SHARPE LLP, 1100 SUPERIOR AVENUE, SEVENTH FLOOR, CLEVELAND, OH, 44114, US

(73) Assignee: IMI TAMI - Institute for Research and Development Ltd., Haifa (IL)

(21) Appl. No.: 11/445,772

(22) Filed: Jun. 2, 2006

(30) Foreign Application Priority Data
Feb. 20, 2006 (IL) 173,815

(51) Int. Cl. A23F 3/16

(52) U.S. Cl. 426/330.3

(57) ABSTRACT

Disclosed herein is a layered edible product comprising a core including a pretreated fruit seed component; and at least one layer of dried fruit component, enveloping, covering, or at least partially diffusing into said core.

### BACKGROUND AND FIELD OF THE INVENTION

The present invention relates generally to an edible, free-flowing, plant-origin product comprising a core composed of a pretreated fruit seed component and at least one layer of a dried fruit component, enveloping, impregnating, covering or contacting with at least a portion of the core. The invention also relates to methods for producing and/or utilizing such a layered edible product.

Previous attempts to spray dry fruit juice components have not resulted in a free-flowing powder suitable for encapsulation, etc. See, for example, US Patent Application Publication No. 20050118312, filed Jun. 2, 2005. A suitable stable-and edible carrier, adapted to effectively load appropriate amounts of either natural or fermented juices to practical concentrations is needed. Moreover, a method of producing a dried fruit product suitable for further processing, e.g., capsulation, tableting, etc., is also desired.

### SUMMARY OF THE INVENTION

The invention described herein pertains to an edible product including a core comprising a pretreated fruit seed component and at least one layer of a dried fruit component, enveloping, impregnating, covering or contacting with at least a portion of the core. The resulting product is free-flowing, plant, such as fruit, origin powder which contains no artificial additives or carriers. Processes for producing and/or utilizing such a product are also disclosed herein.

The core comprises of a natural matrix selected from seed material such as seed cake (i.e., a seed by-product from which seed oil has been removed by crushing or grounding by industrial means), intact or agglomerated seeds, or at least partially chopped seeds. The seed material is pretreated (i.e. dried, pre-milled or milled) to form a powdered core. The powdered core is then immersed in, or enveloped, impregnated, or covered with at least one layer of a dried fruit component. The dried fruit component is a dried liquid matter that is selected from a group of materials including juice, a fermented juice, an extract of peel or a fermentation mixture which includes primarily fruit peel, water, sugar and fermenting yeast; and further wherein the liquid is jellified, immersed, impregnated onto said core, or at least partially diffused within said core. Preferably, at least one of said layers comprises of about 5 to about 30% w/w of the dried fruit fermentation component. For example, fermented pomegranate juice, as set forth in U.S. Pat. No. 5,891,440, the disclosure of which is incorporated herein in its entirety by reference, can be utilized to form the dried fruit component.

It is also within the scope of the present invention wherein the core is provided from plant seeds segments. Moreover, it is acknowledged that least a portion of said core may be milled oil-extracted seeds. Oil-extracted seeds are the by-product of processes such as expeller pressing, supercritical fluid extracting with carbon dioxide, solvent extracting, lyophilizing, etc. The oil-extracted plant seed product may comprise one or more ingredients selected from a group consisting of plant seed cake and especially fruit seed cake, plant seeds and especially fruit seeds, plant seeds powder and especially fruit seeds powder. These materials are treated, preferably ground or milled by one or more steps, to produce a particle size distribution D₅₀ of from about 10 μm to about 40 μm. The materials may be further screened to D₁₀₀=0.2 mm.

Furthermore, at least a portion of the layer of a dried fruit component is processed by a drying, squeezing, extracting, fermenting and/or concentrating a dried liquid matter such as those defined above. Furthermore, the fermented product may comprise fermented juice, fermentation mixture comprising plant peel and especially fruit peel, water, sugar, fermenting yeast or any combination thereof. The enveloping layers may comprise about 5 to about 40% w/w, including from about 15% to about 30% w/w, of the processed fruit juice.

It is also within the scope of the present invention wherein the aforesaid product is especially adapted to be utilized as a food, food ingredient, food supplement, food additive, a cosmetic, a medicament or a pharmaceutical product.

Another object of the invention is to define a novel method of producing an edible, free-flowing, powdered product. The method comprises steps as follows: (a) providing a core comprising a milled fruit seed component; (b) combining the core with a liquid fruit component to produce slurry; and then, (c) drying the slurry so as at least one layer of dried fruit is enveloping said core. Steps (b) and (c) are possibly repeated in the manner the layered product is combined with liquid juice and subsequently dried plurality of times, so as a product characterized by a multiple layers, enveloping the aforesaid core is obtained.

It is further within the scope of the present invention wherein the core comprises a matrix selected from seed cake, agglomerated seeds, at least partially chopped seeds, milled seeds and seed powder. The at least one layer defined above is possibly obtained a liquid matter, selected from a group including juice, a fermented juice, an extract of peel or a fermentation mixture which includes primarily fruit peel, water, sugar and fermenting yeast; and further wherein said liquid is jellified, immersed, impregnated onto said core, or at least partially diffused within said core. At least one of said layers may comprise from about 5 to about 40% w/w of the dried fruit fermentation component. Moreover, the core or the at least one layer of dried fruit is possibly provided in this method from pomegranate seeds or juice, respectively.

These and other non-limiting aspects and/or objects of the development are more particularly disclosed below.

### BRIEF DESCRIPTION OF THE FIGURE

The following is a brief description of the drawing, which is presented for the purposes of illustrating the development disclosed herein and not for the purposes of limiting the same.

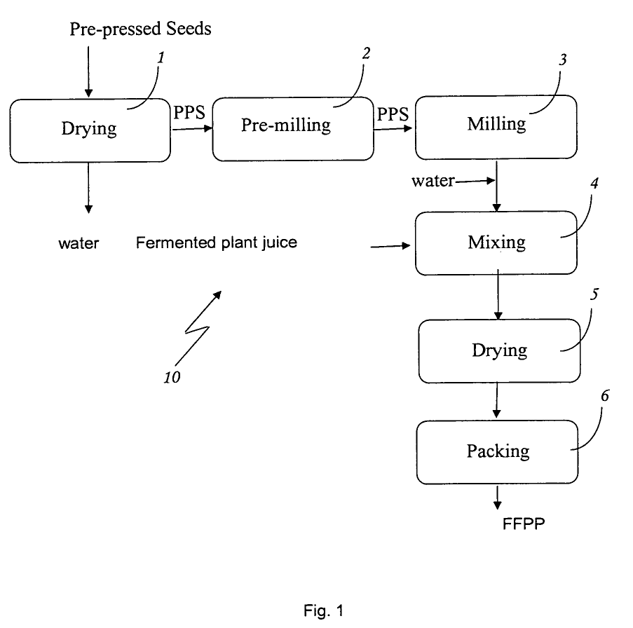

In order to understand the invention and to see how it may be implemented in practice, a preferred embodiments is now be described, by way of non-limiting example only, with reference to the accompanying drawing, in which FIG. 1 discloses a method (10) for obtaining a plant powder in a process comprising a step of spray drying.

### DESCRIPTION OF THE PREFERRED EMBODIMENTS

The following specification sets forth the preferred embodiments of the present invention. The embodiments of the invention disclosed herein are the best modes contemplated by the inventors for carrying out their invention in a commercial environment, although it should be understood that various modifications can be accomplished within the parameters of the present invention to provide a layered edible product including a core comprising pretreated fruit seed; and at least one layer of dried fruit, enveloping the same.

The term “fruit” is used hereinafter to define any single plant-origin composition, to a combination of a plurality (i.e., more than one) fruits, vegetables and/or plants, such as pomegranates, stone fruits, pome fruits, citrus fruits, grapes, berries, melons (e.g. watermelon or cantaloupe), and cucurbits (e.g. zucchini or cucumber).

The disclosed process utilizes a fruit-origin seed carrier to be combined with fruit juice. The combining of said seed core with a liquid fruit component comprises at least one contacting step selected from a group of coating, loading, impregnating, doping, immobilizing, interacting, at least partially solubilizing or dispersing, crystallizing, precipitating or admixing means.

The term ‘fruit seeds’ or shortly ‘seeds’ refers hereinafter to fruits seeds or the like. The seeds may be milled, pressed or otherwise treated before finely drying it. The core of the product is possibly obtained from different fruits or plants or obtained from the same source.

The term ‘layered edible product’ refers hereinafter to a product characterized by a core; the core is at least partially coated, immersed, doped, filled, entrapped, immobilized, sprayed, disposed, soaked, coated, diffused, impregnated, glued, painted and/or enveloped by at least one matrix, especially fruit or plant origin matter, especially a fruit juice that was dried onto and/or into said core.

In accordance with one embodiment, the present invention relates to an industrial process for the production of a layered edible product. For example, fermented fruit juice (FJ) can be loaded on a matrix comprising milled seeds. The concentration of the dry fermented juice in the end product enveloping layers ranges from about 5 to 40% w/w and the product is useful for capsulation in tablets, pills, capsules or the like. The feasibility of the process has been demonstrated on a 2 kg bench scale and on a 100 kg pilot scale.

A spray dry-based method of transforming fermented fruit juice (FJ) into a stable and flowing powder suitable for capsule filling is also disclosed herein. The method comprises inter alia at least two stages of milling of vegetative solids, e.g., seeds. The first pre-milling is provided by a means e.g., of rotor beater mill (Retcsh, Germany); followed by a second milling step, e.g., by utilizing vortex milling means (Super Fine Israel); forming a suspension comprising the same; and then drying it to a free-flowing powder. The method defined above is especially useful in unit operation comprising a spray dryer.

Raw materials for the process comprising fruit solids (i.e., the carrier) and fermented juice (i.e., the loaded liquid). The carrier is pre-pressed fruit solids (PPS) or seeds which contain about half of the initial oil content (e.g., about 15 to about 20% w/w). Seeds with different oil concentrations, such as 0% w/w oil (e.g., pressed and extracted seeds) and 100% w/w oil (e.g., whole seeds) were tested as well. It is in the scope of the present invention and in a non-limiting manner, fermented fruit juice (FJ) loaded liquid comprises about 22% solids (loss on drying, LOD), without ethanol; and about 15% solids (LOD) with about 4 to 9% w/w ethanol. MPPS denotes milled (−pressed) fruit solids.

Reference is now made to FIG. 1, presenting a method (10) for obtaining a plant powder in a process comprising a spray dryer. The method comprising the steps of drying pre-pressed seeds (1), pre-milling the same (2), further milling (3), admixing the same in a fermented plant juice while diluting it with a fluid, especially treated water (4), spray drying the obtained slurry (5) and packaging the same (6) so as a fruit-originated running powder is obtained. More specifically, pressed fruit seeds comprising from 0% to about 50% (wt) of total oil content in whole seeds, e.g., 7.5% (wt) oil, are dried in a dryer, a double cone dryer for example. The dried pressed fruit seeds are pre-milled in a suitable mill, impact mill for example such that dried milled seeds is characterized e.g., by d₁₀₀<0.5 mm is obtained. In a subsequent step or steps of milling, e.g., by a means of a vortex mill, fine powder is obtained, e.g., d₅₀, within the range of about 10 μm to about 40 μm. Effective measure of fluids, e.g., treated water, natural or fermented juice, plant juice comprising about 15% (wt) solids on drying or a mixture thereof are admixed to the powder in a mixer, e.g., high shear mixer, so as to produce a suspension comprising about 5 to about 10% (wt) solids and about 7.5% to about 40% dried juice. The suspension in then dried in a spray dryer. The dried suspension, LOD<1.0%, d₉₀<0.5 mm is obtained. The final fruit layered product may then be packaged for delivery such as placed in capsules, etc.

The present disclosure will further be illustrated in the following non-limiting working examples, it being understood that these examples are intended to be illustrative only and that the disclosure is not intended to be limited to the materials, conditions, process parameters and the like recited herein. All proportions are by weight unless otherwise indicated.

### EXAMPLES

Pressed pomegranate seeds (about quarter of a ton) were dried in double cone dryer (by De Dietrich; Germany) at 80° C. and 60-100 mbar for 12 hours to LOD of about 1%. The seeds were pre-milled to d₁₀₀<0.5 mm by Retsch rotor beater mill (Germany) on 1 mm screen. The pre-milled seeds were then loaded with concentrated pomegranate juice (10% solids) to form a slurry.

Separately, when spray drying is the desired drying process, the spray drying process requires ultra-fine vortex pulverizing. In such a process, the pre-ground seeds from above were further milled by Super Fine (Yokneam, Israel) ultra-fine vortex pulverizing mill to about D₅₀=2.5 μm and screened to D₁₀₀=0.2 mm. The resulting yield was about 90% (D₁₀₀<0.2 mm). The total milling (pre-milling and milling) may be improved by partial recycling of the feed. Those finely milled seed components were then mixed with the juice noted above to form a slurry.

The slurry was dried by spray drying using a Niro (Copenhagen, Denmark) Mobile Minor dryer (bench scale) and a Niro Production Minor (scale up) operating in the range of 350° C. and 110° C., temperatures of inlet and outlet air respectively. The drying capacities of water evaporation were 4 kg/hr water and 22 kg/hr respectively. Residual alcohol was effectively removed by drying. This process produced a free-flowing, layered, edible fruit product comprising a core covered or impregnated with a dried fruit component.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. An edible product comprising a core including a pretreated fruit seed component; and, at least one layer of a dried fruit component at least partially contacting the core.

2. The product according to claim 1, wherein said pretreated fruit seed component comprises seed materials selected from the group consisting of seed cake, intact or agglomerated seeds, at least partially chopped seeds, which have been dried and milled to produce a particle size distribution D₅₀ of from about 10 μm to about 40 μm.

3. The product according to claim 1, wherein dried fruit component is a dried liquid selected from a group consisting of a juice, a fermented juice, an extract of peel or a fermentation mixture which includes fruit peel, water, sugar and fermenting yeast; and further wherein said liquid is jellified, immersed, impregnated onto said core, or at least partially diffused into said core.

4. The product according to claim 1, wherein said layer of a dried fruit component comprises from about 5 to about 40% w/w of a dried fruit fermentation component.

5. The product according to claim 1, wherein said core or said dried fruit component comprises a pomegranate seed or juice.

6. The product according to claim 1, wherein said core comprises oil-extracted plant seeds which have been dried and milled to produce a particle size distribution D₅₀ of from about 10 μm to about 40 μm and screened to D₁₀₀=0.2 mm.

7. The product according to claim 6, wherein said oil-extracted plant seeds comprise one or more ingredients selected from a group consisting of plant seed cake, plant seeds and plant seeds powder.

8. The product according to claim 1, wherein at least a portion of the at least one layer of a dried fruit component is processed by a drying, squeezing, extracting, fermenting or concentrating means.

9. The product according to claim 8, wherein the dried fruit component comprises dried fermented juice, or a fermentation mixture comprising plant peel, water, sugar, fermenting yeast, or any combination thereof.

10. The product according to claim 1, wherein the at least one layer of the dried fruit component comprises from about 5 to about 40% w/w of dried processed fruit juice.

11. The product according to claim 1, adapted to be utilized as a food, food ingredient, food supplement, food additive, a cosmetic, a medicament or a pharmaceutical product.

12. A method for producing an edible, free-flowing, powdered product, the method comprising: (a) providing a core comprising a milled fruit seed component; (b) combining said core with a liquid fruit component to produce a slurry; and (c) drying said slurry so as at least one layer of a dried fruit component is enveloping said core.

13. A method of producing an edible, free-flowing powdered plant-origin product comprising the steps of providing pre-pressed seeds, milling the seeds to a particle size distribution D₅₀ of from about 10 μm to about 40 μm, admixing the same in a fermented plant juice to produce a slurry, and spray drying the slurry to form an edible product.

14. The method according to claim 12, wherein steps (b) and (c) are repeated to produce an edible product characterized by multiple layers enveloping said core.

15. The method according to claim 12, wherein said core comprises seed cake, intact or agglomerated seeds, or at least partially chopped seeds, which have been milled to produce a particle size distribution D₅₀ of 100 μm or less.

16. The method according to claim 12, wherein said liquid fruit component is selected from a group consisting of a juice, a fermented juice, an extract of peel, or a fermentation mixture which comprises fruit peel, water, sugar and fermenting yeast; and further wherein said liquid fruit component is jellified, immersed, impregnated onto said core, or at least partially diffused within said core.

17. The method according to claim 16, wherein at least one of said layers comprises from about 5 to about 40% w/w of a dried fruit fermentation component.

18. The method according to claim 16, wherein said core or said layer of a dried fruit component is provided from pomegranate seeds or juice.

19. An edible product, the product comprising:
(a) a fruit seed component; and
(b) a dried fruit component;
wherein said fruit seed component is impregnated with said dried fruit component.

20. A method of producing an edible product, the method comprising:
(a) providing a fruit seed component;
(b) combining said fruit seed component with a liquid fruit component to produce a slurry; and
(c) drying said slurry to produce the edible product comprising said fruit seed component impregnated with a dried fruit component.
